(19) United States
(12) Plant Patent Application Publication
(10) Pub. No.: US 2002/0108157 P1
(43) Pub. Date: Aug. 8, 2002

(54) Chrysanthemum named Reballet

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/776,924

(22) Filed: Feb. 6, 2001

(51) Int. Cl. A01H 5/00

(52) U.S. Cl. PLT/295

(57) ABSTRACT

A Chrysanthemum plant named Reballet characterized by its medium sized blooms with apricot ray-florets and yellow-green disc florets.

### RELATED APPLICATIONS

The present application is related to the following co-pending applications:

Application no. Filing Date Title

### BACKGROUND OF THE INVENTION

The new plant of the present invention comprises a new and distinct cultivar of Chrysanthemum plant that is a cross of unknown Dendranthema Grandiflora parents. The new and distinct cultivar was a seedling resulting from an open pollination among groups of chrysanthemum cultivars maintained under the control of the inventor for breeding purposes. The new and distinct cultivar was discovered and selected as one flowering plant by Rob Noodelijk in a controlled environment (greenhouse) in Rijsenhout Holland in May 1997. The first act of asexual reproduction of Reballet was accomplished when vegetative cuttings were taken from the initial selection in August 1997 in a controlled environment in Rijsenhout Holland.

### SUMMARY OF THE INVENTION

The present invention is a new and distinct variety of chrysanthemum bearing medium sized blooms with apricot ray-florets and yellow-green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a stem of the cultivar in full bloom.

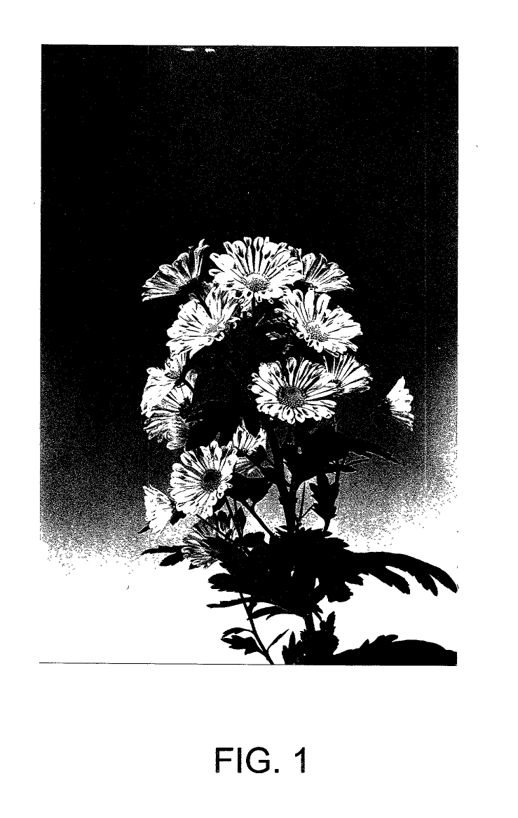

FIG. 2 shows the blooms of the cultivar.

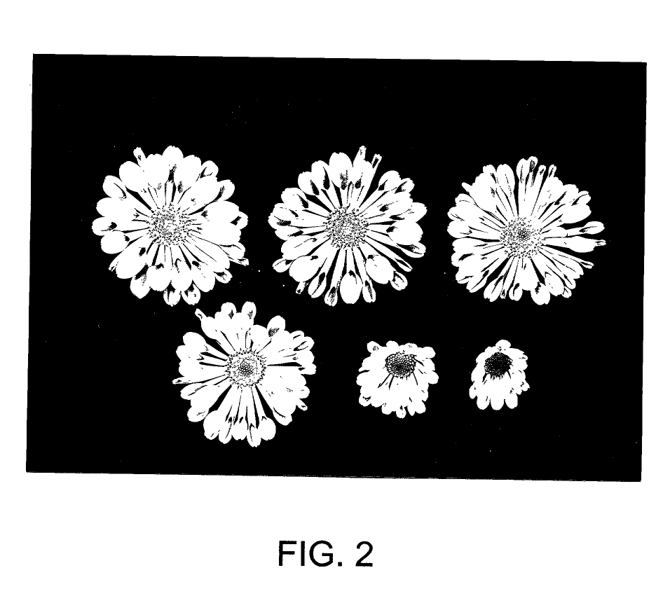

FIG. 3 shows the foliage of the cultivar.

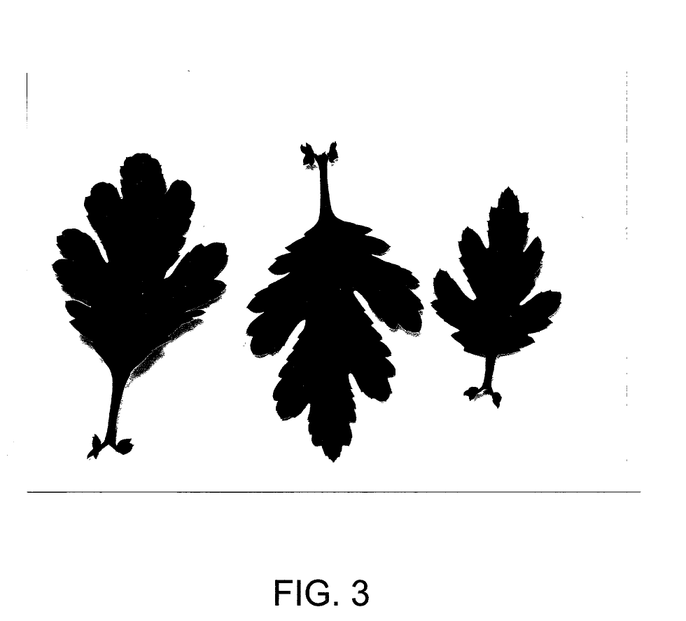

### DESCRIPTION OF THE INVENTION

This new variety of chrysanthemum is of the botanical classification Dendranthema Grandiflora. The observations and measurements were gathered from plants grown in a greenhouse in Rijsenhout Holland in a photo-periodic controlled crop. The photo-periodic response time in this crop was 49 days. This new variety produces medium sized blooms with apricot ray-florets and yellow-green disc-florets blooming on the plant for 21 days. This new variety of chrysanthemum has been found to retain its distinctive characteristics throughout successive propagations however the phenotype may vary significantly with variations in environment such as light intensity and temperature. To show the phenotype as described Reballet may be planted without assimilation lightning between week 50 and 40 under greenhouse conditions in Holland. With assimilation lightning (minimum level 2500 lux) it may be planted year round under greenhouse conditions in Holland.

The following is a description of the plant and characteristics that distinguish Reballet as a new and distinct variety. The color designations are taken from the plant itself. Accordingly, any discrepancies between the color designations and the colors depicted in the photographs are due to photographic tolerances.

Bud

Size.—Medium cross-section +/− 0.8 cm height +/− 1.0 cm.

Outside color.—Greyed-Orange 164 D.

Bloom

Size.—Medium.

Fully expanded.—5,5 cm.

Borne.—Upper portion single flower per peduncle lower portion plural flowers per peduncle.

Number of blooms per single stem.—Average of 11.

Peduncle length.—Near the top +/− 10 cm, near the middle +/− 16 cm, neat the bottom +/− 18 cm.

Peduncle lateral shoot attachment.—Strong.

Peduncle lateral shoot angle.—30°.

Form.—Daisy.

Performance (blooming period).—21 days.

Color

Center of the flower (disc-florets).—Immature yellow-green 151A Mature yellow-green 154A.

Color of upper surface of the majority of the ray-florets.—Yellow-orange 16C.

Color of the lower surface of the majority of the ray-florets.—Greyed-orange 164D.

Tonality from distance.—An apricot daisy with spoon tipped ray-florets.

Discoloration.—None.

Ray Florets

Texture.—Upper and under side smooth.

Number.—36-38.

Cross-section.—Tubular with spatulate end.

Longitudinal axis of majority.—Straight.

Shape of tip.—Spatulate.

Fragrance.—Typical chrysanthemum.

Disc diameter (when visible) 1.2-1.4 cm.

Reproductive Organs

Stamen (present in disc florets only).—Yellow-green, thick +/− 3 mm in length.

Pollen.—Present.

Styles (present in both ray and disc florets).—Yellow-green, thin.

Style length.—+/−-4 mm.

Stigmas.—Yellow-green.

Stigma width.—+/− 2 mm.

Ovaries.—Enclosed in calyx.

Plant

Form.—Spray mum meant for erect culture Herbaceous.

Growth.—Medium.

Height.—100 cm.

Stem color.—Green 138 A.

Stem strength.—Medium.

Stem brittleness.—Absent.

Stem anthocyanin coloration.—Absent.

Internodes.—2 cm.

Flowering response(photo-periodic controlled crop, no natural growing).—21 days.

Foliage

Color.—Upper side green 139A. under side green 138A.

Size.—large; length +/− 10 cm, width+/− 7 cm.

Quantity (number per stem).—28-30.

Shape.—Oval.

Texture.—Fleshy.

Ribs and veins.—Ribs and veins well developed.

Edge.—Crenated.

Shape of base of sinus between lateral lobes.—Round.

Margin of sinus between lateral lobes.—Diverging.

Shape of Base.—Obtuse.

Apex.—Mucronate.

I claim:

1. A new and distinct variety of chrysanthemum plant as described and illustrated.
